(19) United States
(12) Patent Application Publication
(10) Pub. No.: US 2012/0195840 A1
(43) Pub. Date: Aug. 2, 2012

(54) PERSONAL CARE COMPOSITION

(76) Inventors: Ravi Kant Shukla, Bangalore (IN); Vijay Ramachandra Gadgil, Bangalore (IN); Punam Bandyopadhyay, Bangalore (IN); Anil Shankaranarayana Hegde, Bangalore (IN)

(21) Appl. No.: 13/500,441

(22) PCT Filed: Sep. 21, 2010
(86) PCT No.: PCT/EP10/63891

§ 371 Date: Apr. 5, 2012

(30) Foreign Application Priority Data
Oct. 12, 2009 (IN) 2363/MUM/2009

(51) Int. Cl. A61K 8/35; A61Q 19/02; A61Q 17/04

(52) U.S. Cl. 424/59; 424/62

(57) ABSTRACT

The present invention relates to a personal care composition for topical application comprising selective fractions of natural materials having photoprotective and skin lightening applications. The inventors found that certain fractions of natural materials which are rich in anthroquinones or naphthaquinones e.g. those from the Rubiaceae family provide for enhanced broad spectrum photoprotection especially in the visible range while also exhibiting enhanced activity in a biochemical pathway viz. ty-rosinase inhibition which is indicative of improved skin lightening.

### FIELD OF THE INVENTION

The present invention relates to a personal care composition for topical application comprising selective fractions of natural materials having photoprotective and skin lightening applications.

### BACKGROUND OF THE INVENTION

Highly pleasing skin appearance is one of the most desired expectations from personal care products from most consumers around the world. In tropical countries where consumers generally have dark skin, there is a desire to have lighter skin appearance. In consumers who live far from the tropical countries e.g. Caucasian people who generally have lighter skin, there is a need among such consumers to have an even tanned tone of their skin. Any exposure of the skin to sunlight, in such consumers often leads to blotchy skin, referred to as freckles and in some cases they experience hyperpigmentation in localized areas of the skin. Most consumers experience blemishes on their skin after exposure to sun, on healing of wounds or after drying up of acne. In all of the above cases, consumers rely on cosmetic solutions to their skin appearance problems.

Smooth, soft and glowing skin with even skin tone and colour is thus desired by all consumers who use personal care compositions for their skin. To provide this benefit, manufacturers from around the world have tried many approaches. One very commonly used approach is to include sunscreens or sunblocks in such cosmetic products. Sunscreens or sunblocks may be organic compounds or inorganic compounds. Sunscreens are generally organic compounds that work by absorbing ultra-violet (uv) radiation from the sun at a specified wavelength range thus not permitting the uv radiation from reaching the skin surface. UV radiation is believed to be the cause of skin coloration or tanning and if such tanning is uneven, it is disliked by the consumer. Sunblocks are generally inorganic compounds that act as a physical barrier against a wide range of radiation from the sun (both uv and visible light). There are some disadvantages in both these approaches. Organic sunscreens are generally effective only against specific wavelength ranges usually in the uv range i.e. they are not broad spectrum and therefore more than one sunscreen is often used. There are also questions about the stability of these sunscreens on exposure to the sun.

Further it is believed that visible light also contributes substantially to skin darkening or tanning. Most commercially available sunscreens do not provide adequate protection from visible light. Hence there is a need in the art for sunscreen compounds that provide visible light protection in addition to uv protection. Inorganic sunblocks, while being broad spectrum, often are white in colour and leave a pale whitish appearance on the skin which is unnatural and not liked by consumers.

Another approach to controlling the colour, tone and appearance of the skin is the so called skin lightening approach where chemicals are added to personal care compositions which alter the formation of melanin in the skin through biochemical transformation in the stratum corneum thereby changing the colour and appearance of the skin. While this approach has been used successfully in many cosmetic products, they often take time for the desired effect to be visible (in the order of weeks or months). Also researchers are still struggling to improve efficacy of skin lightening agents beyond a certain threshold.

One of the drawbacks of the two approaches (photoprotection and skin lightening) outlined above is that separate actives need to be used to achieve the desired ends. Further, many of the actives for photoprotection and skin lightening are synthetically prepared chemical compounds. Synthetic chemicals have, over time, taken a negative connotation in the consumer's mind. Hence many consumers are more and more preferring actives originating from or extracted from natural sources to be used in such products.

In order to provide a solution to the several drawbacks in the art listed above, the present inventors have been working for many years on deriving actives from natural sources for various personal care benefits. They found to their utter surprise that certain fractions of natural materials which are rich in anthroquinones or naphthaquinones e.g. those from the Rubiaceae family provide for enhanced broad spectrum photoprotection especially in the visible range while also exhibiting enhanced activity in a biochemical pathway viz. tyrosinase inhibition which is indicative of improved skin lightening.

Water extracts of Rubia cordifolia are known to provide skin lightening benefits (WO041057618, Unilever). KR2002079055 discloses a sunscreen composition containing natural materials such as water extracts of Rubia cordifolia. FR2483226 (Pierre Fabre, 1981) discloses a process for the preparation of natural dye starting from an extract of plants from the Rubiaceae family chosen from Rubia tinctorum, Rubia iberica, Coprosma areolata, Coprosma Australis, Coprosma lucida, Coprosma parviflora, Morinda tinctoria, Morinda longiflora, Asperula odorata and Galium desypodum. The extraction process comprises extracting the plant part directly with organic solvents e.g. methanol, ethanol, isopropyl alcohol or acetone. The present inventors have determined that the high level of anthroquinones and napthaquniones in the plant extract, desired to get the benefits of the present invention, are not possible with direct extraction with these organic solvents. Rather, the preferred process is to first extract the plant source with hot water followed by separation of substantially water-insoluble (at 25° C.) ingredients from the aqueous extract.

It is thus an object of the present invention to provide for a personal care composition that comprises fractions obtained from natural sources that provides enhanced photoprotection even over visible light spectrum in addition to the uv range.

It is another object of the present invention to provide for a personal care composition that comprises fractions obtained from natural sources that gives enhanced skin lightening in addition to photoprotection benefits.

### SUMMARY OF THE INVENTION

According to one aspect of the present invention there is provided a personal care composition comprising
(i) 0.001 to 20% by weight an extract of a plant source which comprises anthraquinones or naphthoquinones in an amount of 25 to 100% by weight of the extract; and
(ii) a cosmetically acceptable base.

According to another aspect of the invention, there is provided a process for preparing an extract of a plant source which comprises anthraquinones or naphthoquninones in an amount of 25 to 100% by weight of the extract comprising the steps of;
(i) heating the plant source in water at a temperature in the range of 40 to 100° C.;
(ii) separating the insoluble matter to prepare an aqueous fraction;
(iii) subjecting said aqueous fraction to a step of fractionating substantially water insoluble constituents by any known method to prepare said extract of a plant source.

By water insoluble is meant having a water solubility in the range 5 to 50%, more preferably from 5 to 40% by weight of the extract at 25° C.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

“Personal care composition” as used herein, is meant to include a composition for topical application to skin and/or hair of mammals, especially humans. Such a composition may be generally classified as leave-on or rinse off, and includes any product applied to a human body for improving appearance, cleansing, odor control or general aesthetics. The composition of the present invention can be in the form of a liquid, lotion, cream, foam, scrub, gel, soap bar or toner, or applied with an implement or via a face mask, pad or patch. Non-limiting examples of personal care compositions include leave-on skin lotions and creams, shampoos, conditioners, shower gels, toilet bars, antiperspirants, deodorants, shave creams, depilatories, lipsticks, foundations, mascara, sunless tanners and sunscreen lotions. “Skin” as used herein is meant to include skin on the face and body (e.g. neck, chest, back, arms, underarms, hands, legs, buttocks and scalp). The composition of the invention is also of relevance to applications on any other keratinous substrate of the human body other than skin e.g. hair where products may be formulated with the specific aim of providing photoprotection.

The various natural materials from which the extract may be prepared are those of the Rubiaceae, Liliaceae or Plumbaginaceae family. Of these it is preferred that natural material is from the Rubia cordifolia, Aloe vera or Plumbago zeylanica species. Anthraquinones have the general structural formula:

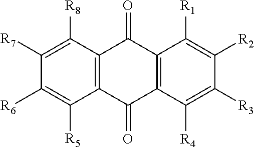

where all the groups R1 to R8 may be H or OH. Naphthoquinones have the general formula:

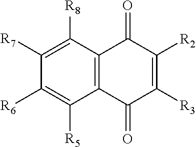

where the groups R2, R3, R5, R6, R7 and R8 may be H or OH.

By the term anthraquinones or naphthoquninones is meant general structures given above and includes derivatives of such structures. Preferred derivatives are the glycosides and prenylated forms. Thus preferred derivatives are;

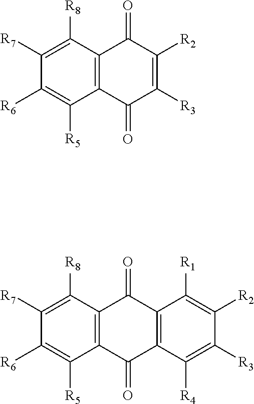

where one or more than one of the groups R1 to R8 may be a glucoside, glycoside, prenyl, —CHO, COOH, NO₃, NH₂, alkyl, acyl, esters, ether or —SH group. Preferably the personal care composition comprises 40 to 95% anthraquinones or naphthoquinones.

According to a preferred aspect of the invention the extract of the plant source has a water solubility in the range of 5 to 50%, more preferably from 5 to 40% by weight of the extract at 25° C. Thus the extract is relatively water insoluble i.e the extract comprises compounds which are relatively hydrophobic. The relative hydrophobicity of the compounds present in the extract of the invention is preferably attained by extracting the compounds in water at a high temperature e.g. from 50 to 100° C., cooling the extract to about 25° C. and further extracting the relatively hydrophobic compounds using known methods hereinafter described. The extract of the plant source is preferably incorporated in 0.01 to 10%, further more preferably in 0.1 to 5% by weight of the composition.

The personal care composition comprises a cosmetically acceptable base. The cosmetically acceptable base is preferably a cream, lotion, gel or emulsion. Personal care compositions may be prepared using different cosmetically acceptable emulsifying or non-emulsifying systems and vehicles. A highly suitable base is a cream. Vanishing creams are especially preferred. Vanishing cream bases generally comprise 5 to 25% be weight of the composition of fatty acid and 0.1 to 10% by weight of the composition of soap. Vanishing cream base gives a highly appreciated matty feel to the skin. C12 to C20 fatty acids are especially preferred in vanishing cream bases, further more preferred being C14 to C18 fatty acids. The most preferred fatty acid is stearic acid. The fatty acid in the composition is more preferably present in an amount in the range of 5 to 20% by weight of the composition. Soaps in the vanishing cream base include alkali metal salt of fatty acids, like sodium or potassium salts, most preferred being potassium stearate. The soap in the vanishing cream base is generally present in an amount in the range of 0.1 to 10%, more preferably 0.1 to 3% by weight of the composition. Generally the vanishing cream base in cosmetic compositions is prepared by taking a desired amount of total fatty matter and mixing with potassium hydroxide in desired amounts. The soap is usually formed in-situ during the mixing.

The composition of the invention may additionally comprise a skin lightening agent. The skin lightening agent is preferably chosen from a vitamin B3 compound or its derivative e.g. niacin, nicotinic acid or niacinamide, or other well known skin lightening agents e.g. aloe extract, ammonium lactate, arbutin, azelaic acid, kojic acid, butyl hydroxy anisole, butyl hydroxy toluene, citrate esters, 3 diphenyl propane derivatives, 2, 5 dihydroxybenzoic acid and its derivatives, ellagic acid, fennel extract, gluco pyranosyl-1-ascorbate, gluconic acid, glycolic acid, green tea extract, hydroquinone, 4 hydroxyanisole and its derivatives, 4-hydroxy benzoic acid derivatives, hydroxycaprylic acid, lemon extract, linoleic acid, magnesium ascorbyl phosphate, mulberry root extract, 2,4 resorcinol derivatives, 3,5 resorcinol derivatives, salicylic acid, vitamins like vitamin B6, vitamin B12, vitamin C, vitamin A, a dicarboxylic acid, resorcinol derivatives, hydroxycarboxylic acid like lactic acid and their salts e.g. sodium lactate, and mixtures thereof. Vitamin B3 compound or its derivative e.g. niacin, nicotinic acid and niacinamide are the more preferred skin lightening agent as per the invention, most preferred being niacinamide. Niacinamide, when used, is preferably present in an amount in the range of 0.1 to 10%, more preferably 0.2 to 5% by weight of the composition.

The personal care composition may preferably additionally comprise one or more uv sunscreens. The uv sunscreens may be inorganic or organic. A wide variety of organic sunscreen agents are suitable for use in combination with the essential ingredients of this invention. Suitable UV-A/UV-B sunscreen agents include 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl-p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid and mixtures thereof. Most suitable organic sunscreens are 2-ethylhexyl-p-methoxycinnamate and butylmethoxydibenzoylmethane. A safe and effective amount of sunscreen may be used in the compositions of the present invention. The composition preferably comprises from about 0.1% to about 10%, more preferably from about 0.1% to about 5% by weight of the composition of a sunscreen agent.

Useful inorganic sun-blocks are also preferably used in the present invention. These include, for example, zinc oxide, iron oxide, silica, such as fumed silica, and titanium dioxide. Ultrafine titanium dioxide in either of its two forms, namely water-dispersible titanium dioxide and oil-dispersible titanium dioxide, is especially suitable for the invention. Water-dispersible titanium dioxide is ultra-fine titanium dioxide, the particles of which are non-coated or which are coated with a material to impart a hydrophilic surface property to the particles. Examples of such materials include aluminium oxide and aluminium silicate. Oil-dispersible titanium dioxide is ultrafine titanium dioxide, the particles of which exhibit a hydrophobic surface property, and which, for this purpose, can be coated with metal soaps such as aluminium stearate, aluminium laurate or zinc stearate, or with organosilicone compounds. By “ultrafine titanium dioxide” is meant particles of titanium dioxide having an average particle size of less than 100 nm, preferably 70 nm or less, more preferably from 10 to 40 nm and most preferably from 15 to 25 nm. By topical application to the skin of a mixture of both water-dispersible ultrafine titanium dioxide and oil-dispersible ultrafine titanium dioxide, synergistically enhanced protection of the skin against the harmful effects of both UV-A and UV-B rays is achievable. Ultrafine titanium dioxide is the preferred inorganic sun-block agent as per this invention. The total amount of sun block that is preferably incorporated in the composition according to the invention is from 0.1 to 5% by weight of the composition.

The composition according to the invention may also comprise other diluents. The diluents act as a dispersant or carrier for other materials present in the composition so as to facilitate their distribution when the composition is applied to the skin. Diluents other than water can include liquid or solid emollients, solvents, humectants, thickeners and powders. Examples of each of these types of vehicle, which can be used singly or as mixtures of one or more vehicles, are as follows: emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate and myristyl myristate; solvents, such as ethyl alcohol, isopropanol, acetone, ethylene glycol monoethyl ether, diethylene glycol monobutyl ether and diethylene glycol monoethyl ether; and powders, such as chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose and ethylene glycol monostearate.

The cosmetically acceptable base is usually from 10 to 99.9%, preferably from 50 to 99% by weight of the composition, and can, in the absence of other cosmetic adjuncts such as a skin lightening agent or a uv sunscreen, form the balance of the composition.

The composition of the invention may comprise a conventional deodourant base as the cosmetically acceptable base. By a deodourant is meant a product in the stick, roll-on, or propellant medium which is used for personal deodourant benefit e.g. application in the under-arm area which may or may not contain anti-perspirant actives. Deodourant compositions can generally be in the form of firm solids, soft solids, gels, creams, and liquids and are dispensed using applicators appropriate to the physical characteristics of the composition.

Deodourant compositions which are delivered through roll-ons generally comprise a liquid carrier. Such a liquid carrier can be hydrophobic or comprise a mixture of both hydrophilic and hydrophobic liquids. They may be in the form of an emulsion or a microemulsion. The liquid carrier or mixture of carriers often constitutes from 30 to 95% by weight of the composition and in many instances from 40 to 80%. Hydrophobic liquid carriers commonly can comprise one or more materials selected within the chemical classes of siloxanes, hydrocarbons, branched aliphatic alcohols, esters and ethers that have a melting point not higher than 25° C. and a boiling point of at least 100° C. Hydrophilic carrier liquids that can be employed in compositions herein commonly comprise water and/or a mono- or polyhydric alcohol or water-miscible homologue. Monohydric alcohols often are short chain, by which is meant that they contain up to 6 carbons, and in practice are most often ethanol or sometimes iso-propanol. Polyhydric alcohols commonly comprise ethylene or propylene glycol, or a homologue can be employed such as diethylene glycol.

The compositions that remain in liquid form can be applied employing conventional applicators such as a roll-on or by being pumped or squeezed through a spray-generating orifice. Such compositions may be thickened, for example, using one or more thickeners described subsequently herein. Compositions that are firm solids, commonly obtained by use of a gellant or structurant, can be applied employing a stick applicator and soft solids, gels and creams can be applied employing an applicator having a dispensing head provided with at least one aperture through which the soft solid, gel or cream can be extruded under mild pressure.

Suitable thickeners or gellants that may be used for achieving this is by use of water-soluble or dispersible materials of higher viscosity, including various of the emulsifiers, and/or thickened or gelled with water-soluble or water-dispersible polymers including polyacrylates, and water-soluble or dispersible natural polymers, such as water-soluble polysaccharide or starch derivatives, such as alginates, carageenan, agarose and water-dispersible polymers include cellulose derivatives. The concentration of such polymers is often selected in the range of from 1 to 20% by weight of the composition, depending on the extent of thickening or structuring required, and the effectiveness of the chosen polymer in the liquid/mixture.

One class of structurant which is desirable by virtue of its long standing proven capability to produce firm solids and more recently in making soft solids comprises waxes. Herein, the term wax is employed to encompass not only materials of natural origin that are solid with a waxy feel and water-insoluble at 30-40° C. but melt at a somewhat higher temperature, typically between 50 and 95° C., such as beeswax, candelilla or carnauba wax, but also materials having similar properties. Such other waxes include hydrocarbon waxes, eg paraffin wax, mineral wax and microcrystalline wax; synthetic waxes, such as polyethylene of 2000 to 10000 daltons; and waxy derivatives or waxy components of natural waxes. Mixtures of materials within each class of gellant/structurant can be employed.

When the antiperspirant composition employed herein comprises an aerosol composition, a propellant is employed, commonly in a weight ratio of antiperspirant composition to propellant of from 95:5 to 40:60, and in many formulations, of from 90:10 to 50:50. The propellant is conveniently a low boiling point material, typically boiling below −5° C., for example an alkane such as propane, butane or isobutane, and possibly containing a fraction of pentane or isopentane, or a hydrofluorocarbon or fluorocarbon of similar carbon content. During filling of the aerosol canister, the propellant gas is liquified by virtue of the elevated pressure that is generated therein.

The compositions of the present invention can comprise a wide range of other optional components. The CTFA Cosmetic Ingredient Handbook, Second Edition, 1992, which is incorporated by reference herein in its entirety, describes a wide variety of non-limiting cosmetic and pharmaceutical ingredients commonly used in the skin care industry which are suitable for use in the compositions of the present invention. Examples include antioxidants, binders, biological additives, buffering agents, colorants, thickeners, polymers, astringents, fragrance, humectants, opacifying agents, conditioners, exfoliating agents, pH adjusters, preservatives, natural extracts, essential oils, skin sensates, skin soothing agents and skin healing agents.

The composition is formulated in any known format, more preferred formats being creams or lotions.

### Process

The invention also provides for a process for preparation of an extract of a plant source which comprises anthraquinones or naphthaquinones in an amount of 25 to 100% by weight of the extract. The preferred process has the following steps:

(i) Extraction: The soluble matter from the plant source is first extracted in water by heating it in water at a temperature in the range of 40 to 100° C., more preferably in the range of 50 to 95° C. The various parts of the plant which may be used for extraction include leaves, root or stem, preferably the stem or root more preferably stem. The extraction is preferably carried out for 2 to 8 hours, more preferably 4 to 6 hours.
(ii) Separation of insolubles: The insoluble matter from the extraction process i.e from the leaves, root or stem, is then separated from the mixture. The solution containing the water extract is the aqueous fraction of the plant source. This fraction is preferably dried to a powder by separating out the water preferably by evaporation, before further processing steps are carried out.
(iii) Fractionation: The aqueous fraction is then further fractionated to prepare a fraction having relatively water insoluble constituents i.e. constituents that have a water solubility in the range of 5 to 50%, more preferably from 10 to 40% by weight of the extract at 25° C. Processes that may be used to prepare this relatively water insoluble (relatively hydrophobic) fraction are summarized below:
(a) Separation using MCl gel. The separation is done through a polystyrene based polymeric resin (MCl gel), e.g. one obtained from Supelco. Typical process is as follows: solution of aqueous extract is loaded onto a glass column packed with MCl gel (pre-washed with ethanol or methanol and swelled in water for 2-4 h). MCl gel is a hydrophobic gel which tends to adsorb and retain hydrophobic fractions which can be further eluted with solvents of varying polarity. The process is generally done at around room temperature e.g. in the range of 20 to 30° C. The preferred mobile phase is methanol. It is preferred that the MCl gel adsorbed fraction which is separated using the MCl gel described above is further fractionated using mid-polar solvents. Preferred mid polar solvents are ethyl acetate, acetone, chloroform, dichloromethane, hexanes, toluene, xylene, diethyl ether, butanol and, isobutyl methyl ketone. The most preferred mid-polar solvent is ethyl acetate.
(b) Separation by precipitation using mid-polar solvents: An alternate process to prepare the desired fraction comprises the process of precipitation which involves the step of taking the powder form of the aqueous fraction and adding a polar to mid-polar solvent at around room temperature viz. 20 to 30° C. The weight ratio of aqueous fraction to solvent is preferably from 1:1 to 1:5. Preferred polar to mid-polar solvent includes methanol, ethanol, propanol, butanol and acetone. This aqueous fraction may be further fractionated with mid-polar solvents like ethyl acetate, acetone, chloroform, dichloromethane, hexanes, toluene, xylene, diethyl ether, butanol and isobutyl methyl ketone. The most preferred mid-polar solvents is ethyl acetate. The desired fraction is obtained by evaporation of soluble solvent phase.
(c) Separation using acid hydrolysis: Yet another preferred process to prepare the desired fraction comprises the step of acid hydrolysis of the powder form of the aqueous fraction of the plant material. An aqueous solution of extract is acidified with dilute mineral acids (e.g. hydrochloric acid or sulphuric acid) to bring the pH in the range of about 2 to 5. The preferred pH is in the range of 3 to 4. The acid hydrolysis is preferably done at room temperature viz. 20 to 30° C. for about 1 to 5 hours. The precipitate obtained after acid hydrolysis is washed with water. The precipitate is then preferably dried under vacuum to obtain the desired fraction.

According to yet another aspect of the present invention there is provided use of an extract of a plant source which comprises anthraquinones or naphthoqinones in an amount of 25 to 100% by weight of the extract as a skin lightening agent. The use is preferably non-therapeutic.

According to yet another aspect of the invention there is provided use of an extract of a plant source which comprises anthraquinones or naphthoqinones in an amount of 25 to 100% by weight of the extract as a photoprotective agent. The use is preferably non-therapeutic.

The specific fraction for use either as a sunscreen or as a photoprotective agent is further characterized by having a solubility in water in the range of 5 to 50% by weight of the extract at 25° C. This fraction is preferably a hydrophobic fraction of the aqueous extract. This fraction is preferably prepared by a process of the present invention.

The invention is now further described by way of the following non-limiting examples.

### EXAMPLES

### Example 1

### Aqueous Extract of Rubia Cordifolia

Twigs of the herb Rubia Cordifolia (sourced from Channabasappa & Co., Bangalore) were first powdered using a Retsch trade mill. To 100 g of the powdered herb, one liter of distilled water was added and was refluxed for 8 h and then filtered hot through muslin cloth. The Filtrate was evaporated by using a rotaevaporator at 45° C. under reduced pressure to get a powder form of the aqueous extract of the herb.

### Example 2

### Methanol Soluble Fraction of Rubia Cordifolia

About 10 g of the aqueous extract as prepared in example 1 was dissolved in about 1 liter of distilled water to obtain a clear solution. This solution was loaded onto MCl gel (100 g) packed column. The MCl gel used herein was polystyrene-divinylbenzene resin purchased from Supelco. The loaded material was then eluted with water (1 L) and methanol (1.5 L). Both fractions were dried on rotaevaporator at 45° C. under reduced pressure. Methanol eluted fraction (˜1 g) is the sample used in this example.

### Example 3

### Ethyl Acetate Soluble Fraction of Rubia Cordifolia

About 0.5 g of the methanol fraction obtained from example 2 was loaded onto a silica column and then sequentially eluted with hexane, ethyl acetate and methanol. All of the fractions were dried on a rotaevaporator at 45° C. under reduced pressure. The yields were ˜0.05 g of hexane soluble fraction, ˜0.1 g of ethyl acetate soluble fraction and ˜0.26 g of methanol soluble fraction. The ethyl acetate soluble fraction was the sample as per this example.

The w/w solubility in water (at 25° C.) of each of the abovementioned examples was determined. The percentage w/w of anthraquinones and naphthaquinones in each of the examples was also determined using well known chromatographic techniques like HPTLC and HPLC.

The results are summarized in table 1.

TABLE 1

| | % w/w solubility in water | % w/w anthraquinones |
|---|---|---|
| Example | (25° C.) | and naphthaquinones |
| 1 | ~100% | 5-15% |
| 2 | 30 (+/−10)% | 40-75% |
| 3 | 10 (+/−8)% | 70-95% |

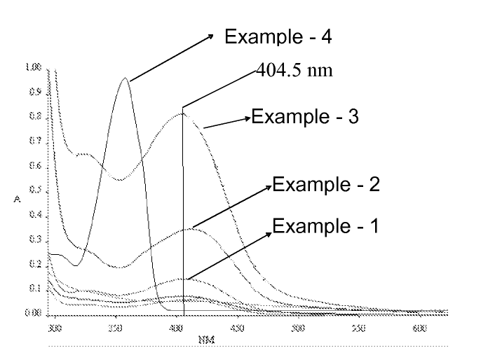

The uv absorption spectra of the examples are presented in FIG. 1. In the FIGURE, example 1 was measured at a concentration of 1000 ppm in water while examples 2 and 3 were measured at 100 ppm in methanol. Example 4 is the absorption spectra of Parsol 1789 at a concentration 10 ppm in methanol. The data in table 1 read along with the spectra in FIG. 1 indicates that samples as per the invention (examples 2 and 3) provide vastly improved UV-visible protection as compared to an aqueous extract of the plant material. Further the samples as per the invention provide broader spectrum protection (in visible region in addition to uv region) as compared to a commercially available sunscreen (Parsol 1789).

An indication of the efficacy of skin lightening was determined using an in-vitro tyrosinanse inhibition assay using mushroom tyrosinase. The assay comprised measuring O-diphenolase activities of mushroom tyrosinase with L-DOPA as substrate. This was determined spectrophotometrically by measuring the rate of dopachrome formation at 450 nm (ε=3700 M−1 cm−1). Inhibition studies were done by measuring the dopachrome formation in the presence of various extracts of plant material. The experiment was conducted using the following procedure:

The following stock solutions were prepared. 0.1 M phosphate buffer (KH2PO4, pH=6.5); 3.5 mM L-DOPA in phosphate buffer; 1 mg/ml mushroom tyrosinase enzyme (100 kU) in phosphate buffer. To the KH₂PO₄ buffer (q.s. to 200 μL) in a 96 well plate having flat bottom was added the desired sample (about 6 μl) followed by 8 μL of mushroom tyrosinase enzyme. This was left at room temperature in the dark for 15 min to enable incubation. Then L-DOPA (57 μL, 10 mM) was added to the resultant solution and the progress of dopachrome formation was observed by measuring the absorbance at 450 nm (A450) on a Tecan spectrophotometer. Percent inhibition was calculated using (Control: without inhibitor)

% Inhibition=[(ΔA₄₅₀Control−Δ_{A450}Sample)Δ_{A450}Control]×100.

The results for examples 1 and 2 are set fourth in table 2. Both examples were used at 900 ppm concentration. Additionally, a sample as per example 2 was used to measure tyrosinase inhibition except that it was carried out at 450 ppm (example 2A).

TABLE 2

| | % mushroom tyrosinase |
|---|---|
| Example | inhibition |
| Example 1 | 18 |
| Example 2 | 45 |
| Example 2A | 24 |

Example 2 (at 10 ppm) was tested with human tyrosinase enzyme in accordance with the procedure set forth hereinabove for the mushroom tyrosinase and the efficacy of this was compared to ethyl resorcinol at 7.5 ppm concentration (example 5). Ethyl resorcinol is known to be one of the best tyrosinase inhibitor. The data on % human tyrosinase inhibition is shown in table 3.

TABLE 3

| | % human tyrosinase |
|---|---|
| Example | inhibition |
| Example 2 | 46 |
| Example 5 | 40 |

### Examples 6 and 7

### Personal Care Compositions Comprising Plant Extracts

Personal care compositions for photoprotection of the skin were prepared as summarized in table 4.

TABLE 4

| | Example 6 | Example 7 |
|---|---|---|
| Ingredient | weight % | weight % |
| Extract of Rubia cordifolia as per example 1 | 4.0 | 0.0 |
| Extract of Rubia cordifolia as per example 3 | 0.0 | 1.0 |
| (ethyl acetate soluble fraction) | | |
| Stearic acid | 13.4 | 13.4 |
| Potassium stearate | 5.2 | 5.2 |
| Glycerine | 1.0 | 1.0 |
| Niacinamide | 1.0 | 1.0 |
| Cetyl alcohol | 0.5 | 0.5 |
| Isopropyl myristate | 0.8 | 0.8 |
| Dimethicone | 0.5 | 0.5 |
| Micronised titanium dioxide (Tayca MT | 0.2 | 0.2 |
| 100Z ™) | | |
| Methyl paraben | 0.2 | 0.2 |
| Propyl paraben | 0.1 | 0.1 |
| Perfume | 0.3 | 0.3 |
| Water | To 100 | To 100 |

The above compositions were prepared as follows. Water phase ingredients (water, glycerine and potassium hydroxide) was heated to 75° C. in a water bath and molten stearic acid was added to the water phase with stirring (at 1500 rpm) until an emulsion was formed. Remaining ingredients (except niacinamide and micronised titanium dioxide) were heated to 60° C. (oil phase) and added to the above emulsion under stirring at 2000 rpm. After 10 minutes of mixing, the emulsion was allowed to cool under constant stirring until the formation of a cream. Micronised titanium dioxide and niacinamide were added at 50° C., and perfume added at 40° C. Aqueous extract (example 1) of Rubia Cordifolia (in example 6) was added in the water phase whereas the ethyl acetate soluble fraction (example 3) of Rubia Cordifolia (in example 7) was added in the oil phase in the corresponding compositions. The uv and visible absorption provided by the compositions of examples 6 and 7 were measured. The procedure used was as follows: 100 mg of the composition was dissolved in 25 ml of ethanol in a volumetric flask. In the case of example 6 the sample was further diluted with ethanol (by a factor of 4) to make up to 100 ml to enable measurement of absorption at the same total active concentration (of Rubia Cordifolia extract). UV absorption measurements on the samples thus prepared were carried out on a Perkin Elmer spectrophotometer. The absorbance was measured at representative wavelengths of the UV-B, UV-A, and visible regions viz. at 320 nm, 360 nm and 420 nm respectively. Table 5 summarises the data.

TABLE 5

| | Concen- | Absorption | Absorption | Absorp- |
|---|---|---|---|---|
| | tration of | UVB | UVA | tion |
| Example | extract (ppm) | (320 nm) | (360 nm) | Vis (420 nm) |
| Example 6 | 40 | 0.106 | 0.055 | 0.035 |
| Example 7 | 40 | 0.302 | 0.197 | 0.155 |

The data in table 5 indicates that a personal care composition as per the invention (example 7) provides for vastly improved photoprotection over a wide wavelength range as compared to compositions that comprise conventional aqueous extract.

### Example 8

### Deodourant Composition as Per the Invention

Personal care composition in the form of a deo-stick for additionally providing skin lightening benefit was prepared as summarized in table 6. The procedure to prepare the stick was as follows:

Propylene glycol and PEG 200 were heated to 70-80° C. Sodium stearate powder was then added to this mixture and stirred to dissolve it. The rest of the ingredients including the samples as per example 3 were then added to prepare the final molten mixture. The mixture was then poured into tubes of desired sizes. The tubes were then refrigerated after closing the lid of the tubes for one to two hours. The deo-stick was then ejected from the tubes.

TABLE 6

| | % by weight |
|---|---|
| Ingredient | Example 8, wt % |
| Sample as per example 3 | 2 |
| Propylene glycol | 25 |
| Polyethylene glycol | 30 |
| Sodium stearate | 4 |
| Disodium EDTA | 0.02 |
| AMP 95 | 0.5 |
| BHT | 0.01 |
| Dextrin | 2.5 |
| Water | To 100 |

The present invention thus provides for a personal care composition that comprises fractions obtained from natural sources that give enhanced photoprotection even over visible light spectrum in addition to the uv range. Further, the composition gives enhanced skin lightening, in addition to photoprotection benefits.

1. A personal care composition comprising
(i) 0.001 to 20% by weight an extract of a plant source which comprises anthraquinones or naphthaquinones in an amount of 25 to 100% by weight of the extract; and
(ii) a cosmetically acceptable base.

2. A composition as claimed in claim 1 wherein said anthraquinones or naphthaquinones is in a prenylated form or a glycosidated form.

3. A composition as claimed in claim 1 or claim 2 wherein said extract comprises 40 to 95% anthraquinones or naphthaquinones.

4. A composition as claimed in any one of the preceding claims wherein said extract has a solubility in water in the range of 5 to 50% by weight of the extract at 25° C.

5. A composition as claimed in claim 4 wherein said solubility is in the range of 5 to 40% by weight of the extract at 25° C.

6. A composition as claimed in any one of the preceding claims wherein said plant source is from the family of Rubiaceae.

7. A composition as claimed in claim 6 wherein said plant source is Rubia cordifolia.

8. A process for preparing an extract of a plant source which comprises, anthraquinones or naphthaquinones in an amount of 25 to 100% by weight of the extract comprising the steps of:
(i) heating the plant source in water at a temperature in the range of 40 to 100° C.;
(ii) separating the insoluble matter to prepare an aqueous fraction;
(iii) subjecting said aqueous fraction to a step of fractionating substantially water insoluble constituents by any known method to prepare said extract of a plant source.

9. A process as claimed in claim 8 wherein said step of fractionating said substantially water insoluble constituents comprises any one of the methods selected from (i) separation using MCl gel (ii) separation by precipitation using mid-polar solvents including ethyl acetate, acetone, chloroform, dichloromethane, hexanes, toluene, xylene, diethyl ether, butanol or isobutyl methyl ketone or (iii) separation using a chemical step involving acid hydrolysis to separate out the insoluble matter.

10. A process as claimed in claim 9 wherein said step of separation using MCl gel is followed by a step of solubilising said substantially water insoluble constituents in a mid-polar solvent selected from ethyl acetate, acetone, chloroform, dichloromethane, hexanes, toluene, xylene, diethyl ether, butanol or isobutyl methyl ketone and separating the solvent from the solution.

11. A process as claimed in claim 9 or 10 said mid-polar solvent is ethyl acetate.

12. A process as claimed in any one of the preceding claims 8 to 11 wherein said aqueous fraction in step (ii) is dried to a powder before carrying out the rest of the process steps.

13. Use of an extract of a plant source which comprises anthraquinones or naphthoqinones in an amount of 25 to 100% by weight of the extract as a skin lightening agent.

14. Use of an extract of a plant source which comprises anthraquinones or naphthoqinones in an amount of 25 to 100% by weight of the extract as a photoprotective agent.
